(19) United States
(12) Patent Application Publication
(10) Pub. No.: US 2019/0216718 A1
(43) Pub. Date: Jul. 18, 2019

(54) COSMETIC COMPOSITION CONTAINING COMPLEX EXTRACT OF SARGASSUM HORNERI AND ENTEROMORPHA PROLIFERA

(71) Applicant: Sang Hwang Mi-in Co., Ltd., Busan (KR)

(72) Inventors: Young-sang Shin, Busan (KR); Chang-wook Shin, Busan (KR); Young-woo Shin, Busan (KR); Yu-ham Park, Busan (KR)

(73) Assignee: Sang Hwang Mi-in Co., Ltd.

(21) Appl. No.: 15/880,796

(22) Filed: Jan. 26, 2018

(30) Foreign Application Priority Data
Jan. 16, 2018 (KR) 10-2018-0005691

(51) Int. Cl. A61K 8/9722; A61K 8/9711; A61Q 19/08

(52) U.S. Cl. CPC A61K 8/9722; A61K 2800/5922; A61Q 19/08; A61K 8/9711

(57) ABSTRACT

A cosmetic composition containing a complex extract of Sargassum horneri and Enteromorpha prolifera, which is capable of exhibiting antioxidant activity. The cosmetic composition of the invention contains a complex extract of Sargassum horneri and Enteromorpha prolifera as an active ingredient.

### REFERENCE TO RELATED APPLICATIONS

This application claims the priority benefit of Korean Patent Application No. 10-2018-0005691 filed on Jan. 16, 2018, the entire contents of which are incorporated herein by reference.

### FIELD OF THE INVENTION

The present invention relates to a cosmetic composition, and more particularly to a cosmetic composition containing a complex extract of Sargassum horneri and Enteromorpha prolifera, which is capable of exhibiting antioxidant activity.

### BACKGROUND OF THE INVENTION

The sea is a veritable hearth of life, in which many living organisms live, and it is the largest library of living information on the planet, containing therein a wide variety of kinds of physiologically active substances.

While the physiologically active ingredients derived from terrestrial organisms have already been studied due to their ease of accessibility, research into the physiologically active ingredients of marine organisms, which are relatively inaccessible, is still insufficient.

Unlike land ecosystems, marine ecosystems have a high salt content and an environment characterized by limited oxygen supply, and thus the ecological defense mechanisms of living organisms are very different from those of terrestrial organisms. For this reason, various physiologically active substances are receiving interest as raw materials, and in recent years, intensive research and development for the exploration and production of new materials has been carried out in some advanced countries.

Despite advances in the extraction and separation of active ingredients from natural substances and the emergence of advanced analytical instruments, few marine organisms have been systematically studied in connection with natural ingredients and physiological activities.

Marine organisms, especially seaweeds, are capable of absorbing ultraviolet light in response to damage to tissues and cells due to ultraviolet light and dry environments, but also produce various antioxidants to cope with secondary damage caused by ultraviolet light. Most of them have various defense mechanisms, including the production of moisturizing components such as polysaccharides and the like, and thus carry out life activities, making it possible to develop functional materials with high added value utilizing the self-defense components of these seaweeds.

Marine organisms that harm human life or property are called harmful marine organisms. Such harmful marine organisms, such as Sargassum horneri, Enteromorpha prolifera, and the like, which have been introduced in large amounts domestically from the East China Sea coast in southern China because of an increase in seawater temperature due to global warming, are expected to continue to proliferate in the future.

These harmful marine plants may be utilized as edible products, fertilizers, and the like, but they proliferate on a large scale, accumulate on the shore, generate offensive odors, and cause damage to farms. Moreover, Sargassum horneri and Enteromorpha prolifera, deposited thereon, rot and cause offensive odors due to the lack of collection staff and budget.

For example, in 2015, the amounts of Sargassum horneri and Enteromorpha prolifera collected due to massive inflows from the East China Sea coast in southern China were recorded at 9,850 tons and 10,000 tons, respectively, not all of which could be collected owing to budget shortages and lack of labor.

Although these harmful marine species, Sargassum horneri and Enteromorpha prolifera, are obvious choices for the development and commercialization of high-value-added products based on the latest biotechnology relative to their potential value, they are merely collected and thus discarded, or are used as fertilizer.

Meanwhile, recent lifestyle trends favoring health and relaxation have become popular, and moreover, the desire to be beautiful creates market demands, and thus functional cosmetics are receiving great attention.

Functional cosmetics have now evolved beyond simple cosmetic concepts, and the concepts of prevention of aging and the treatment of diseases have been introduced, and demand for the development of new materials capable of performing whitening, wrinkle reduction, and ultraviolet blocking, which are thus required to exhibit high functionality and versatility, is increasing.

In particular, the quality of life pursued by middle-aged and elderly people is expected to further increase the demand for functional cosmetics in the future due to changes in consumers' desires and values.

In addition, the cosmetics industry is the second-largest industry after the pharmaceutical industry in the fine chemical industry, but relatively few technologies have been introduced thereto, and 50% or more of raw materials for cosmetics is dependent on foreign imports.

In the case of raw materials for cosmetics, the rate of increase in imports is accelerating year by year. It is particularly urgent to secure raw materials for domestic bio-resources in accordance with the Nagoya Protocol of 2014.

Accordingly, there is an urgent need for technology that is able to develop and commercialize a functional cosmetic composition, which may satisfy the activity required of cosmetics, including antioxidant activity for the inhibition of reactive oxygen species related to the endogenous aging of the skin, using seaweeds, for example, brown algae such as Sargassum horneri and green algae such as Enteromorpha prolifera, and which may also exhibit high percutaneous absorption efficiency using a variety of formulation processes.

### SUMMARY OF THE INVENTION

Accordingly, the present invention has been made keeping in mind the problems encountered in the related art, and the present invention is intended to provide a cosmetic composition containing a complex extract of Sargassum horneri and Enteromorpha prolifera, which may exhibit antioxidant activity.

Therefore, the present invention provides a cosmetic composition, containing, as an active ingredient, a complex extract of Sargassum horneri and Enteromorpha prolifera.

Preferably, the complex extract is obtained by mixing Sargassum horneri with Enteromorpha prolifera and then performing ethanol extraction.

Preferably, the complex extract is obtained through extraction with ethanol having a concentration of 40 to 60% at 50 to 70° C. for 1 to 3 hr.

Preferably, the complex extract comprises Sargassum horneri and Enteromorpha prolifera, which are mixed at a weight ratio of 0.5 to 1.2:0.5 to 1.2.

According to the present invention, the cosmetic composition containing a complex extract of Sargassum horneri and Enteromorpha prolifera can exhibit the antioxidant activity required of cosmetics, and can thus be effectively applied to various functional cosmetics such as a sunscreen, a skin, a lotion, a cream, a nutrition pack, a skin cleanser, a mask pack, and the like.

### BRIEF DESCRIPTION OF THE DRAWINGS

FIGS. 1A and 1B show Sargassum horneri and Enteromorpha prolifera according to a preferred embodiment of the present invention;

FIGS. 2A, 2A′, 2B and 2B′ show photographs of hydrothermal extraction according to a preferred embodiment of the present invention;

FIGS. 3A, 3A′, 3B and 3B′ show photographs of ethanol extraction according to a preferred embodiment of the present invention;

FIGS. 4A, 4A′, 4B and 4B′ show photographs of acid hydrolysis according to a preferred embodiment of the present invention;

FIGS. 5A, 5A′, 5B and 5B′ show photographs of enzyme hydrolysis according to a preferred embodiment of the present invention;

FIG. 6 is a graph showing antioxidant activity through ethanol extraction according to a preferred embodiment of the present invention;

FIG. 7 is a graph showing antioxidant activity through acid hydrolysis according to a preferred embodiment of the present invention;

FIG. 8 is a graph showing antioxidant activity through enzyme hydrolysis according to a preferred embodiment of the present invention;

FIG. 9 is a standard curve for measuring total polyphenol content according to a preferred embodiment of the present invention;

FIG. 10 is a graph showing the total phenolic compound content through ethanol extraction according to a preferred embodiment of the present invention;

FIG. 11 is a graph showing the total phenolic compound content through acid hydrolysis according to a preferred embodiment of the present invention;

FIG. 12 is a graph showing the total phenolic compound content through enzyme hydrolysis according to a preferred embodiment of the present invention;

FIG. 13 is a graph showing the dependency on the concentration of an extraction solvent according to a preferred embodiment of the present invention;

FIG. 14 is a graph showing the dependency on the extraction time according to a preferred embodiment of the present invention;

FIG. 15 is a graph showing the dependency on the extraction temperature according to a preferred embodiment of the present invention;

FIG. 16 is a comparative graph showing the dependency on the extraction temperature according to a preferred embodiment of the present invention;

FIG. 17 is a graph showing the cytotoxicity on animal cells according to a preferred embodiment of the present invention; and

FIG. 18 is a graph showing the cytotoxicity on human-derived cells according to a preferred embodiment of the present invention.

### DETAILED DESCRIPTION OF THE INVENTION

Before the description of the present invention, Sargassum horneri and Enteromorpha prolifera, which are harmful to domestic marine ecosystems and the fish industry, are collected in amounts of about ten thousand tons per year, and have been used to date as edible products or fertilizers, but the extent of use thereof is insignificant. Most is discarded.

Hence, in the present invention, functional materials are developed by searching for physiologically active substances of Sargassum horneri and Enteromorpha prolifera, the amounts of which are increasing yearly, and preferred embodiments of the present invention will be described in detail with reference to the accompanying drawings.

<Evaluation of Sample Yield>

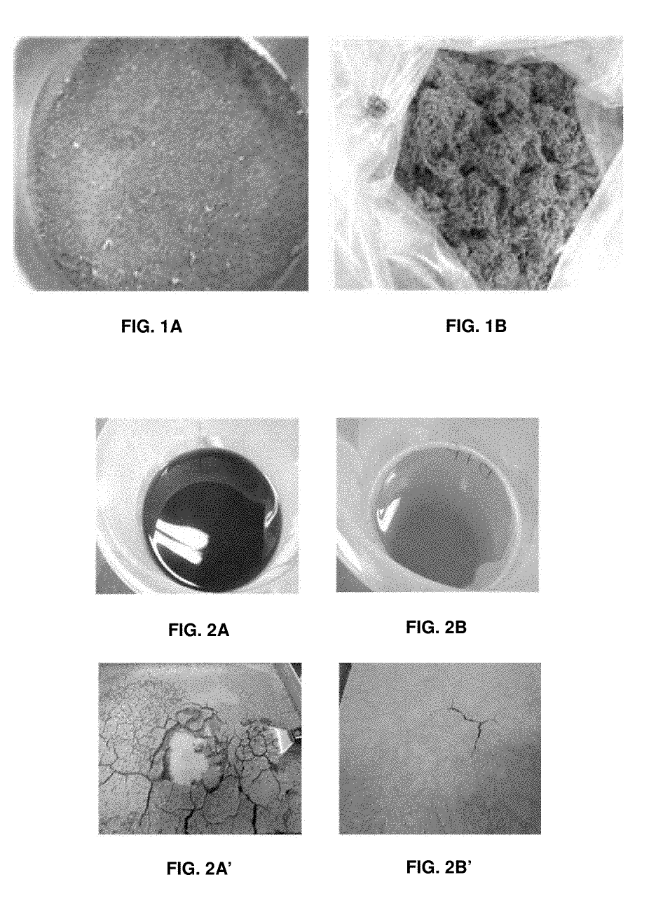

FIGS. 1A and 1B show Sargassum horneri and Enteromorpha prolifera according to a preferred embodiment of the present invention, FIG. 1A illustrating pulverized Sargassum horneri and FIG. 1B illustrating pulverized Enteromorpha prolifera. With reference to FIGS. 1A and 1B, Sargassum horneri, collected from Jeju Province, and Enteromorpha prolifera, collected from Jindo County, were washed three times with flowing water, dried at 60° C. for 24 hr using a hot air dryer, and then pulverized to a size of 100 mesh.

The extraction conditions of the samples were investigated in order to obtain high yield, and hydrothermal extraction, ethanol extraction, acid hydrolysis, and enzyme hydrolysis were conducted in the present invention.

Hydrothermal Extraction

FIGS. 2A, 2A′, 2B and 2B′ shows photographs of hydrothermal extraction according to a preferred embodiment of the present invention. FIG. 2A illustrates the supernatant collected after hydrothermal extraction of Sargassum horneri, and FIG. 2A′ illustrates the dry product obtained by lyophilizing the supernatant of FIG. 2A. Also, FIG. 2B illustrates the supernatant collected after hydrothermal extraction of Enteromorpha prolifera, and FIG. 2B′ illustrates the dry product obtained by lyophilizing the supernatant of FIG. 2B.

With reference to FIGS. 2A, 2A′, 2B and 2B′, each of Sargassum horneri (1 kg) and Enteromorpha prolifera (1 kg) was added with water at a ratio of 1:10 (w/v) and extracted at 80° C. for 3 hr, and the extracts were centrifuged at 8,000 rpm for 20 min, thus collecting respective supernatants, which were then concentrated using a rotary vacuum filter, ultimately obtaining lyophilized products.

Based on the results of hydrothermal extraction, the dry yield of Sargassum horneri was found to be 9.2% and the dry yield of Enteromorpha prolifera was found to be 11.4%.

Ethanol Extraction

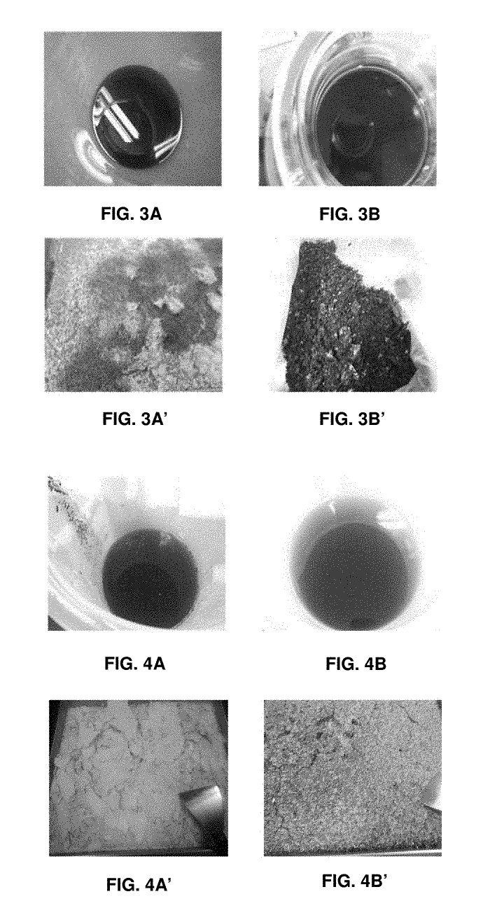

FIGS. 3A, 3A′, 3B and 3B′ show the photographs of ethanol extraction according to a preferred embodiment of the present invention. FIG. 3A illustrates the supernatant collected after ethanol extraction of Sargassum horneri, and FIG. 3A′ illustrates the dry product obtained by lyophilizing the supernatant of FIG. 3A. Also, FIG. 3B illustrates the supernatant collected after ethanol extraction of Enteromorpha prolifera, and FIG. 3B′ illustrates the dry product obtained by lyophilizing the supernatant of FIG. 3B.

With reference to FIGS. 3A, 3A′, 3B and 3B′, each of Sargassum horneri (1 kg) and Enteromorpha prolifera (1 kg) was mixed with 80% ethanol at a ratio of 1:10 (w/v) and extracted at 60° C. for 2 hr, and the extracts were centrifuged at 8,000 rpm for 20 min, thus collecting respective supernatants, after which the collected supernatants were concentrated using a rotary vacuum filter to remove ethanol therefrom, ultimately obtaining lyophilized products.

Based on the results of ethanol extraction, the dry yield of Sargassum horneri was 4.2% and the dry yield of Enteromorpha prolifera was 4.6%, both of which were similarly low. This is deemed to be because the yield is lowered due to the loss of sugar during centrifugation.

Acid Hydrolysis

FIGS. 4A, 4A′, 4B and 4B′ show the photographs of acid hydrolysis according to a preferred embodiment of the present invention. FIG. 4A illustrates the supernatant collected after acid hydrolysis of Sargassum horneri, and FIG. 4A′ illustrates the dry product obtained by lyophilizing the supernatant of FIG. 4A. Also, FIG. 4B illustrates the supernatant collected after acid hydrolysis of Enteromorpha prolifera, and FIG. 4B′ illustrates the dry product obtained by lyophilizing the supernatant of FIG. 4B.

With reference to FIGS. 4A, 4A′, 4B and 4B′, each of Sargassum horneri (1 kg) and Enteromorpha prolifera (1 kg) was mixed with 2 M hydrochloric acid (HCl) at a ratio of 1:10 (w/v) and allowed to react at 121° C. for 20 min and thus hydrolyzed, thereby producing hydrolysates. Thereafter, the pH thereof was adjusted to 5 using sodium hydroxide (NaOH) as a base. The hydrolysates at a pH of 5 were centrifuged at 8,000 rpm for 20 min, thus collecting respective supernatants, which were then concentrated using a rotary vacuum filter, ultimately obtaining lyophilized products.

Based on the results of acid hydrolysis, the dry yield of Sargassum horneri was 27.0% and the dry yield of Enteromorpha prolifera was 40.9%. Here, the yield of Enteromorpha prolifera was as high as about 1.5 times the yield of Sargassum horneri.

Enzyme Hydrolysis

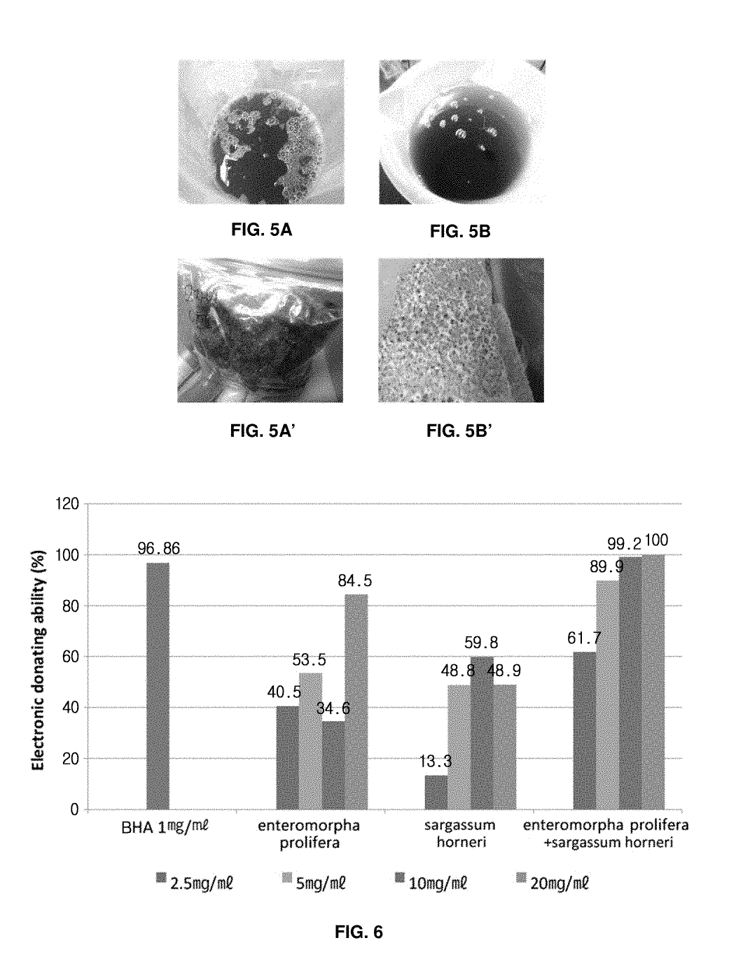

FIGS. 5A, 5A′, 5B and 5B′ show the photographs of enzyme hydrolysis according to a preferred embodiment of the present invention. FIG. 5A illustrates the supernatant collected after enzyme hydrolysis of Sargassum horneri, and FIG. 5A′ illustrates the dry product obtained by lyophilizing the supernatant of FIG. 5A. Also, FIG. 5B illustrates the supernatant collected after enzyme hydrolysis of Enteromorpha prolifera, and FIG. 5B′ illustrates the dry product obtained by lyophilizing the supernatant of FIG. 5B.

With reference to FIGS. 5A, 5A′, 5B and 5B′, each of Sargassum horneri (1 kg) and Enteromorpha prolifera (1 kg) was mixed with 36 Unit Celluclast at a ratio of 1:10 (w/v) and thus treated with the enzyme at 45° C. for 36 hr, thereby producing hydrolysates, which were then centrifuged at 8,000 rpm for 20 min, thus collecting respective supernatants, which were then concentrated using a rotary vacuum filter, ultimately obtaining lyophilized products.

Based on the results of enzyme hydrolysis, the yield of Sargassum horneri was 29.0% and the yield of Enteromorpha prolifera was 37.3%. As in acid hydrolysis, the yield of Enteromorpha prolifera was as high as about 1.4 times the yield of Sargassum horneri.

The yield results of Sargassum horneri and Enteromorpha prolifera depending on the extraction process are shown in Table 1 below.

TABLE 1

| | Sargassum horneri | | | | Enteromorpha prolifera | | | |
|---|---|---|---|---|---|---|---|---|
| | Dry collected | | | | Dry collected | | | |
| | amount (g) | | | Average | amount (g) | | | Average |
| Classification | 1^{st} | 2^{nd} | 3^{rd} | yield (%) | 1^{st} | 2^{nd} | 3^{rd} | yield (%) |
| Hydrothermal | 94 | 90 | 92 | 9.2 | 110 | 118 | 115 | 11.4 |
| extraction | | | | | | | | |
| Ethanol extraction | 38 | 44 | 43 | 4.2 | 50 | 42 | 47 | 4.6 |
| Acid hydrolysis | 286 | 254 | 271 | 27.0 | 425 | 387 | 415 | 40.9 |
| Enzyme hydrolysis | 284 | 287 | 298 | 29.0 | 368 | 370 | 382 | 37.3 |

As is apparent from Table 1, Sargassum horneri exhibited the highest yield upon enzyme hydrolysis, and the greatest yield thereof was obtained, in descending order, from enzyme hydrolysis, acid hydrolysis, hydrothermal extraction, and ethanol extraction. Also, Enteromorpha prolifera exhibited the highest yield upon acid hydrolysis, and the greatest yield thereof was obtained, in descending order, from acid hydrolysis, enzyme hydrolysis, hydrothermal extraction, and ethanol extraction.

For reference, the low yield upon ethanol extraction is considered to be due to the generation of precipitates after the extraction and the loss of sugar caused by centrifugation.

<Evaluation of Antioxidant Activity>

As described above, Sargassum horneri exhibited the highest extraction yield upon enzyme hydrolysis, and Enteromorpha prolifera exhibited the highest extraction yield upon acid hydrolysis.

Based on these results, a Sargassum horneri extract, an Enteromorpha prolifera extract, and a complex extract of Sargassum horneri and Enteromorpha prolifera mixed together were analyzed for antioxidant activity.

Measurement of DPPH-Radical-Scavenging Ability

In order to evaluate antioxidant activity, DPPH-radical-scavenging ability was measured.

To determine the antioxidant capacity, which indicates how much oxidized DPPH (1,1-diphenyl-2-picrylhydrazyl) may be reduced, the antioxidant activity of an Enteromorpha prolifera extract, a Sargassum horneri extract, and a complex extract of Enteromorpha prolifera and Sargassum horneri mixed together was measured.

Specifically, 100 μl of a solution of each of the Enteromorpha prolifera extract, the Sargassum horneri extract, and the complex extract at different concentrations (2.5, 5, 10, 20 mg/ml) was added with 0.9 ml of a 5×10⁻⁴ M DPPH (1,1-diphenyl-2-picrylhydrazyl) solution. The resulting mixture was allowed to stand for 20 min, and the absorbance thereof was measured at 540 nm using a microplate reader (Wallac 1420, USA) in order to determine the concentration of residual radicals. The test control was BHA (1 mg/ml).

For reference, electron donation ability for inhibiting aging by donating electrons to free radicals was represented by a decrease in absorbance of the groups added and not added with the sample solution. (Equation 1 below)

Electron donation ability (%)=[(blank group−reaction group)/blank group]×100 (Equation 1)

Blank group: Absorbance of control

Reaction group: Absorbance of test group

The results of measurement of the DPPH-radical-scavenging ability of the Enteromorpha prolifera sample, the Sargassum horneri sample, and the mixed sample of Enteromorpha prolifera and Sargassum horneri depending on the extraction process are shown in Table 2 below.

TABLE 2

| | | | | | | | | Enteromorpha prolifera + | | |
|---|---|---|---|---|---|---|---|---|---|---|
| | | Enteromorpha prolifera | | | Sargassum horneri | | | Sargassum horneri | | |
| BHA | | | Acid | Enzyme | | Acid | Enzyme | | Acid | Enzyme |
| 1 mg/ml | | EtOH | hydrolysis | hydrolysis | EtOH | hydrolysis | hydrolysis | EtOH | hydrolysis | hydrolysis |
| 96.86 | 2.5 mg/ml | 40.5 | 32.4 | 0.0 | 13.3 | 0.0 | 33.0 | 61.7 | 51.2 | 38.5 |
| | 5 mg/ml | 53.5 | 36.5 | 0.0 | 48.8 | 0.0 | 38.0 | 89.9 | 48.4 | 57.9 |
| | 10 mg/ml | 34.6 | 38.5 | 0.0 | 59.8 | 5.1 | 38.4 | 99.2 | 43.1 | 61.3 |
| | 20 mg/ml | 84.5 | 37.5 | 26.4 | 48.9 | 36.7 | 41.1 | 100.0 | 29.6 | 52.8 |

FIG. 6 is a graph showing the results of antioxidant activity due to ethanol extraction according to a preferred embodiment of the present invention. With reference to FIG. 6, as is apparent from Table 2, each of the Enteromorpha prolifera sample, the Sargassum horneri sample, and the mixed sample of Enteromorpha prolifera and Sargassum horneri was extracted with ethanol (EtOH), and then the DPPH-radical-scavenging ability thereof was measured to determine antioxidant activity.

As seen in the graph of FIG. 6, the blue bar shows the electron donation ability of control BHA (1 mg/ml), the red bar shows the electron donation ability at a concentration of 2.5 mg/ml, the light green bar shows the electron donation ability at a concentration of 5 mg/ml, the violet bar shows the electron donation ability at a concentration of 10 mg/ml, and the bluish green bar shows the electron donation ability at a concentration of 20 mg/ml.

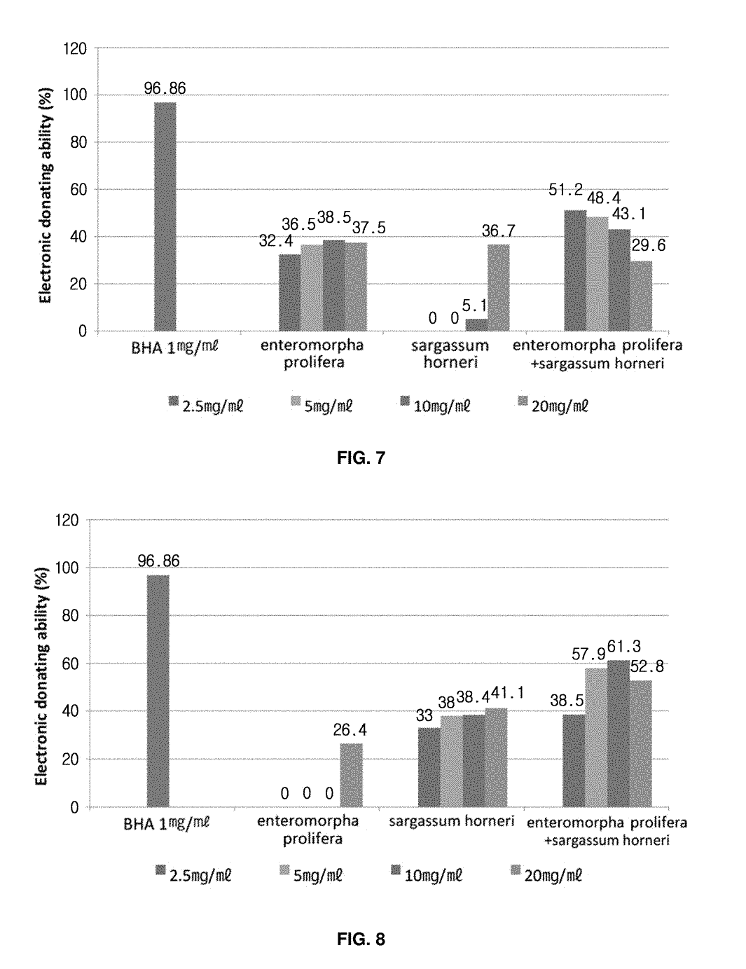

FIG. 7 is a graph showing the results of antioxidant activity due to acid hydrolysis according to a preferred embodiment of the present invention. With reference to FIG. 7, as is apparent from Table 2, each of the Enteromorpha prolifera sample, the Sargassum horneri sample, and the mixed sample of Enteromorpha prolifera and Sargassum horneri was subjected to acid hydrolysis, and then the DPPH-radical-scavenging ability thereof was measured to determine antioxidant activity.

As seen in the graph of FIG. 7, the blue bar shows the electron donation ability of control BHA (1 mg/ml), the red bar shows the electron donation ability at a concentration of 2.5 mg/ml, the light green bar shows the electron donation ability at a concentration of 5 mg/ml, the violet bar shows the electron donation ability at a concentration of 10 mg/ml, and the bluish green bar shows the electron donation ability at a concentration of 20 mg/ml.

FIG. 8 is a graph showing the results of antioxidant activity due to enzyme hydrolysis according to a preferred embodiment of the present invention. With reference to FIG. 8, as is apparent from Table 2, each of the Enteromorpha prolifera sample, the Sargassum horneri sample, and the mixed sample of Enteromorpha prolifera and Sargassum horneri was subjected to enzyme hydrolysis, and then the DPPH-radical-scavenging ability thereof was measured to determine antioxidant activity.

As seen in the graph of FIG. 8, the blue bar shows the electron donation ability of control BHA (1 mg/ml), the red bar shows the electron donation ability at a concentration of 2.5 mg/ml, the light green bar shows the electron donation ability at a concentration of 5 mg/ml, the violet bar shows the electron donation ability at a concentration of 10 mg/ml, and the bluish green bar shows the electron donation ability at a concentration of 20 mg/ml.

Based on the results of measurement of electron donation ability, the complex extract having a concentration of 10 to 20 mg/ml through ethanol extraction of the mixed sample of Enteromorpha prolifera and Sargassum horneri can be concluded to exhibit high activity similar to the control BHA (1 mg/ml).

Measurement of Total Polyphenol Content

In order to evaluate antioxidant activity, total polyphenol content was measured.

Polyphenol is one of the secondary metabolites widely distributed in plants, and exhibits an antioxidant effect by removing reactive oxygen species in a manner in which hydrogen atoms are supplied to reactive free radicals to produce stable non-radicals.

The total phenolic compound was quantified in accordance with a Folin-Denis method in a manner in which 0.5 ml of the sample was added with 0.5 ml of a Folin and Ciocalteu's phenol reagent (Sigma-Aldrich), and after 3 min, the resulting mixture was added with 0.5 ml of 10% sodium carbonate (Na₂CO₃). The resulting mixture was allowed to stand at room temperature for 1 hr, after which the absorbance thereof was measured at 700 nm using a UV/VIS spectrophotometer (Ultrospec 6300 Pro, Amersham Biosciences, Orsay Cedex, France).

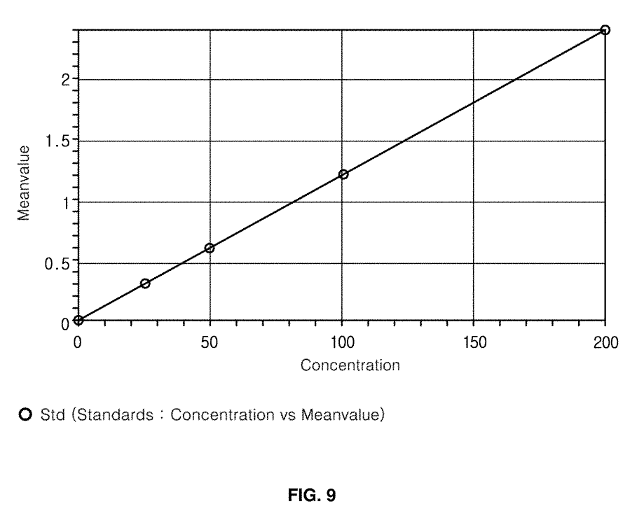

FIG. 9 is a standard curve for measuring the total polyphenol content according to a preferred embodiment of the present invention. With reference to FIG. 9, a standard curve (at concentrations of 0, 25, 50, 100, 200 μg/ml) was made through analysis using tannic acid (Sigma-Aldrich) as a standard material in the same manner as in the sample, and based thereon, the total phenolic compound content per g of the sample was calculated. The results are given in Table 3 below.

TABLE 3

| Total | | | | | | | Enteromorpha prolifera + | | |
|---|---|---|---|---|---|---|---|---|---|
| polyphenol | Enteromorpha prolifera | | | Sargassum horneri | | | Sargassum horneri | | |
| content | | Acid | Enzyme | | Acid | Enzyme | | Acid | Enzyme |
| (μg/ml) | EtOH | hydrolysis | hydrolysis | EtOH | hydrolysis | hydrolysis | EtOH | hydrolysis | hydrolysis |
| 2.5 mg/ml | 47.71 | 173.17 | 18.57 | 53.03 | 7.71 | 67.17 | 58.40 | 60.86 | 47.46 |
| 5 mg/ml | 96.20 | 351.83 | 29.77 | 97.51 | 18.83 | 140.29 | 102.54 | 162.09 | 84.17 |
| 10 mg/ml | 178.40 | 657.23 | 51.63 | 168.94 | 39.20 | 239.83 | 185.11 | 325.74 | 145.69 |
| 20 mg/ml | 313.06 | 1364.60 | 81.77 | 325.31 | 69.69 | 402.06 | 344.69 | 705.29 | 243.63 |

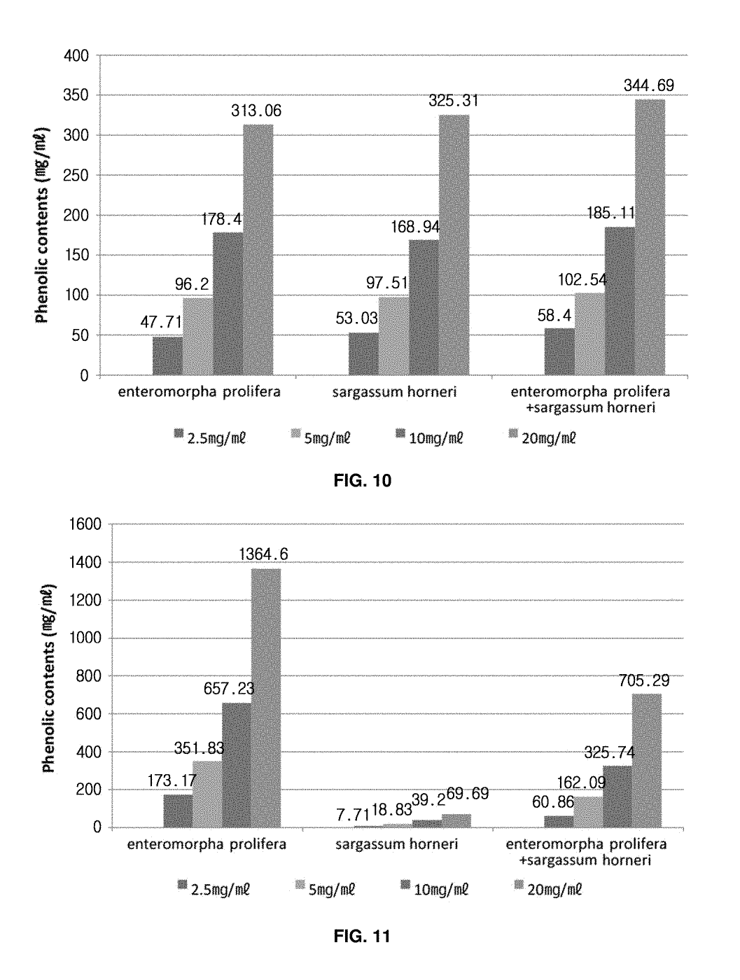

FIG. 10 is a graph showing the results of total phenolic compound content due to ethanol extraction according to a preferred embodiment of the present invention. With reference to FIG. 10, as is apparent from Table 3, each of the Enteromorpha prolifera sample, the Sargassum horneri sample, and the mixed sample of Enteromorpha prolifera and Sargassum horneri was extracted with ethanol (EtOH), after which the total polyphenol content thereof was measured.

As seen in the graph of FIG. 10, the red bar shows the total polyphenol content at a concentration of 2.5 mg/ml, the light green bar shows the total polyphenol content at a concentration of 5 mg/ml, the violet bar shows the total polyphenol content at a concentration of 10 mg/ml, and the bluish green bar shows the total polyphenol content at a concentration of 20 mg/ml.

FIG. 11 is a graph showing the results of total phenolic compound content due to acid hydrolysis according to a preferred embodiment of the present invention. With reference to FIG. 11, as is apparent from Table 3, each of the Enteromorpha prolifera sample, the Sargassum horneri sample, and the mixed sample of Enteromorpha prolifera and Sargassum horneri was extracted through acid hydrolysis, after which the total polyphenol content thereof was measured.

As seen in the graph of FIG. 11, the red bar shows the total polyphenol content at a concentration of 2.5 mg/ml, the light green bar shows the total polyphenol content at a concentration of 5 mg/ml, the violet bar shows the total polyphenol content at a concentration of 10 mg/ml, and the bluish green bar shows the total polyphenol content at a concentration of 20 mg/ml.

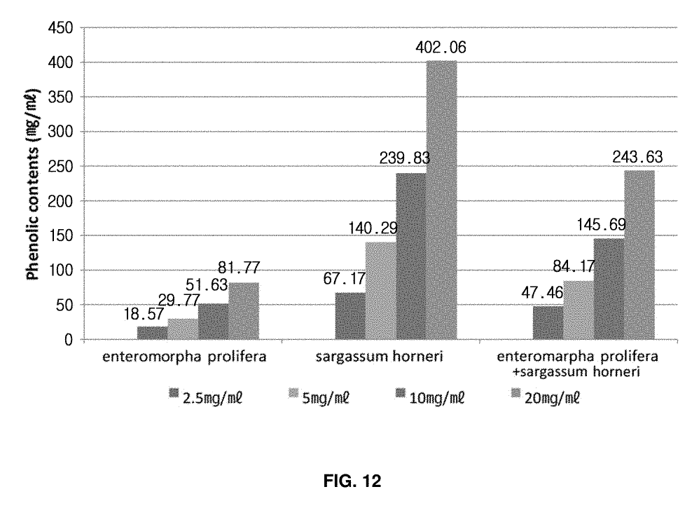

FIG. 12 is a graph showing the results of total phenolic compound content due to enzyme hydrolysis according to a preferred embodiment of the present invention. With reference to FIG. 12, as is apparent from Table 3, each of the Enteromorpha prolifera sample, the Sargassum horneri sample, and the mixed sample of Enteromorpha prolifera and Sargassum horneri was extracted through enzyme hydrolysis, after which the total polyphenol content thereof was measured.

As seen in the graph of FIG. 12, the red bar shows the total polyphenol content at a concentration of 2.5 mg/ml, the light green bar shows the total polyphenol content at a concentration of 5 mg/ml, the violet bar shows the total polyphenol content at a concentration of 10 mg/ml, and the bluish green bar shows the total polyphenol content at a concentration of 20 mg/ml.

Based on the results of measurement of total polyphenol content, the complex extract obtained through acid hydrolysis of the mixed sample of Enteromorpha prolifera and Sargassum horneri had a total polyphenol content of 60.86 at a concentration of 2.5 mg/ml, 162.09 at a concentration of 5 mg/ml, 325.74 at a concentration of 10 mg/ml, and 705.29 at a concentration of 20 mg/ml, and thus manifested the greatest activity compared to the results of ethanol extraction and enzyme hydrolysis.

The total polyphenol content of the complex extract obtained through acid hydrolysis was the highest, but acid hydrolysis causes problems that deteriorate economical operation, such as the corrosion of drums used for acid hydrolysis.

For this reason, the complex extract having a concentration of 10 to 20 mg/ml through ethanol extraction of the mixed sample of Enteromorpha prolifera and Sargassum horneri had a total polyphenol content of 180 to 345 mg/g (185.11 at a concentration of 10 mg/ml, 344.69 at a concentration of 20 mg/ml), and ethanol extraction manifested the second highest total polyphenol content compared to acid hydrolysis, and furthermore, did not cause problems pertaining to economical operation, and is thus very preferable.

Upon the ethanol extraction of the mixed sample of Enteromorpha prolifera and Sargassum horneri, the DPPH-radical-scavenging ability and the total polyphenol content are increased, whereby there are no problems pertaining to economical operation, and superior antioxidant activity may result.

<Establishment of Ethanol Extraction Conditions>

Based on the aforementioned <Evaluation of antioxidant activity>, the antioxidant activity of the complex extract obtained through ethanol extraction of the mixed sample of Enteromorpha prolifera and Sargassum horneri was confirmed to be the greatest.

Accordingly, in order to investigate the detailed conditions for ethanol extraction with the best antioxidant activity, taking into consideration the active ingredient efficacy relative to the optimal yield, optimization of the extraction process was deduced as follows.

1) Optimization of Extraction Process Depending on Ethanol Concentration

Device used: Extraction concentrator (for lab), High-performance liquid chromatography (HPLC), Inductively coupled plasma mass spectrometer (ICP-MS)

Feed: Sargassum horneri (500 kg), Enteromorpha prolifera (500 kg)

Detailed conditions: Ethanol concentration conditions of 20%, 30%, 50%, and 70%

Main contents

Mixing of each sample with ethanol under concentration conditions at a ratio of 1:10 (w/v) and extraction at an inlet temperature of 60° C. for 2 hr

Measurement of the sugar concentration (Brix) of the extracted sample using a sugar meter

A total of 8 runs (samples (two)×concentration conditions (four conditions): 8 runs)

Analysis of the amounts of active ingredient (Fucoidan) and heavy metal (As, Hg, Cd) in the extracted sample

(Active ingredient analyzer: HPLC, Heavy metal analyzer: ICP-MS)

2) Optimization of Extraction Process Depending on Extraction Time

Device used: Extraction concentrator (for lab), HPLC, ICP-MS

Feed: Sargassum horneri, Enteromorpha prolifera

Detailed conditions: Extraction time conditions of 2, 4, 6, and 8 hr

Main contents

Mixing of each sample with ethanol having an optimal concentration at a ratio of 1:10 (w/v) and extraction at an inlet temperature of 60° C. under extraction time conditions of 2, 4, 6, and 8 hr

Measurement of the sugar concentration (Brix) of the extracted sample using a sugar meter

Analysis of the amounts of active ingredient (Fucoidan) and heavy metal (As, Hg, Cd) in the extracted sample

A total of 8 runs (samples (two)×time conditions (four conditions): 8 runs)

3) Optimization of Extraction Process Depending on Extraction Temperature

Device used: Extraction concentrator (for lab), HPLC, ICP-MS

Feed: Sargassum horneri, Enteromorpha prolifera

Detailed conditions: Extraction temperature conditions of 60° C. and 80° C.

Main contents

Mixing of each sample with ethanol having an optimal concentration at a ratio of 1:10 (w/v) and extraction at inlet temperatures of 60° C. and 80° C. under conditions of optimal extraction time

Measurement of the sugar concentration (Brix) of the extracted sample using a sugar meter

Analysis of the amounts of active ingredient (Fucoidan) and heavy metal (As, Hg, Cd) in the extracted sample

A total of 4 runs (samples (two)×temperature conditions (two conditions): 4 runs)

Establishment of a technique for removing heavy metal using a filtration process using the sample extracted under the optimal extraction conditions

4) Optimization of Extraction Process Depending on the Concentration of the Extraction Solvent

TABLE 4

| | | Ethanol | | Extraction | Sugar |
|---|---|---|---|---|---|
| | | concen- | Extraction | temperature | content |
| No. | Sample | tration (%) | time (hr) | (° C.) | (Brix) |
| 1 | Enteromorpha | 20 | 2 | 60 | 10 |
| | prolifera | 30 | 2 | 60 | 13 |
| | | 50 | 2 | 60 | 15 |
| | | 70 | 2 | 60 | 13 |
| 2 | Sargassum | 20 | 2 | 60 | 11 |
| | horneri | 30 | 2 | 60 | 13 |
| | | 50 | 2 | 60 | 15 |
| | | 70 | 2 | 60 | 10 |

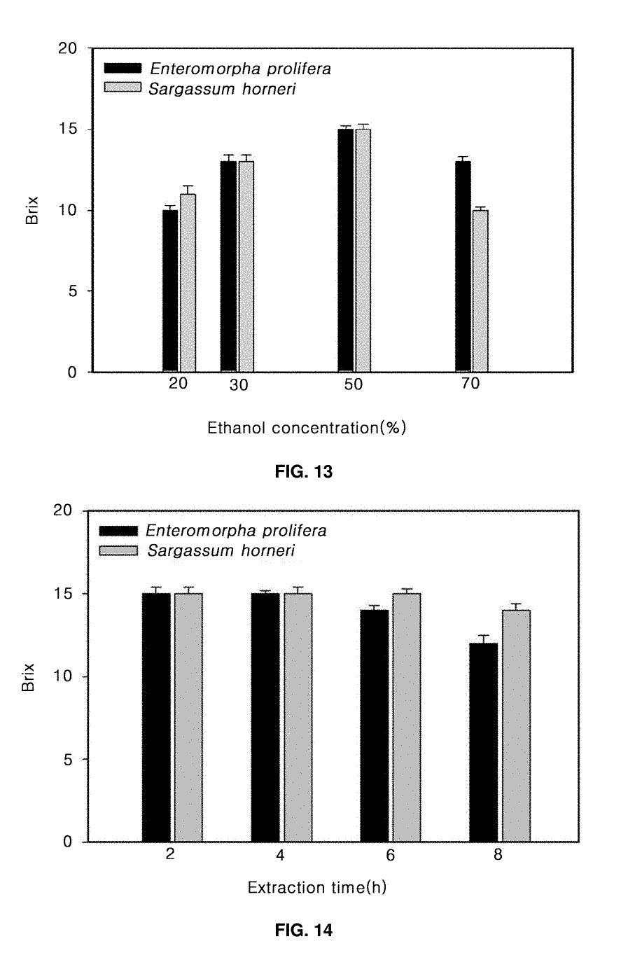

FIG. 13 is a graph showing the dependency on the concentration of the extraction solvent according to a preferred embodiment of the present invention, and shows the results of Table 4. Based on the results of the extraction process depending on the concentration of ethanol (extraction solvent) using Enteromorpha prolifera and Sargassum horneri under the condition that the extraction time and temperature were fixed, the sugar content was the highest, to the level of 15 Brix, at an ethanol concentration of 50%.

5) Optimization of Extraction Process Depending on Extraction Time

TABLE 5

| | | Ethanol | | Extraction | Sugar |
|---|---|---|---|---|---|
| | | concen- | Extraction | temperature | content |
| No. | Sample | tration (%) | time (hr) | (° C.) | (Brix) |
| 1 | Enteromorpha | 50 | 2 | 60 | 15 |
| | prolifera | 50 | 4 | 60 | 15 |
| | | 50 | 6 | 60 | 15 |
| | | 50 | 8 | 60 | 14 |
| 2 | Sargassum | 50 | 2 | 60 | 15 |
| | horneri | 50 | 4 | 60 | 15 |
| | | 50 | 6 | 60 | 14 |
| | | 50 | 8 | 60 | 12 |

FIG. 14 is a graph showing the dependency on the extraction time according to a preferred embodiment of the present invention, and shows the results of Table 5. Based on the results of testing of the optimization of the extraction process depending on the extraction time using Enteromorpha prolifera and Sargassum horneri under the condition that the ethanol concentration was fixed to 50% showing the highest Brix and the extraction temperature was fixed, there was no significant difference, but the sugar content was gradually decreased over time.

6) Optimization of Extraction Process Depending on Extraction Temperature

TABLE 6

| | | Ethanol | | Extraction | Sugar |
|---|---|---|---|---|---|
| | | concen- | Extraction | temperature | content |
| No. | Sample | tration (%) | time (hr) | (° C.) | (Brix) |
| 1 | Enteromorpha | 50 | 2 | 60 | 15 |
| | prolifera | 50 | 2 | 80 | 10 |
| 2 | Sargassum | 50 | 2 | 60 | 15 |
| | horneri | 50 | 2 | 80 | 13 |

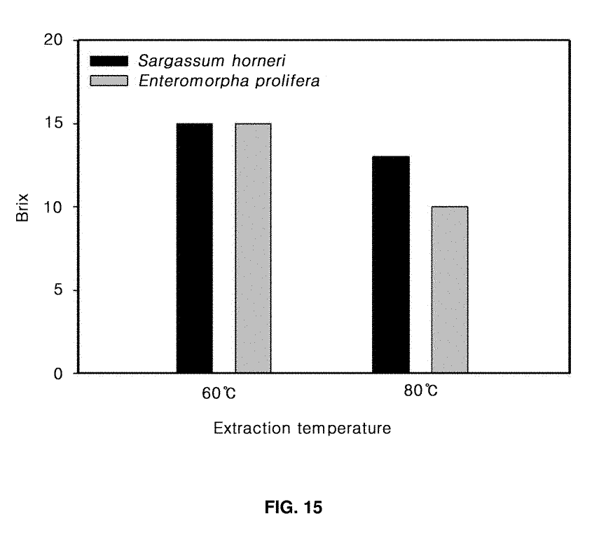

FIG. 15 is a graph showing the dependency on the extraction temperature according to a preferred embodiment of the present invention, and shows the results of Table 6. Based on the results of testing of the optimization of the extraction process depending on the extraction temperature using Enteromorpha prolifera and Sargassum horneri under conditions of 50% ethanol concentration and an extraction time of 2 hr, the sugar content was measured to be lower at 80° C. than at 60° C.

7) Results of Use of Complex Extract of Enteromorpha prolifera and Sargassum horneri

TABLE 7

| | | Ethanol | | Extraction | Sugar |
|---|---|---|---|---|---|
| | | concen- | Extraction | temperature | content |
| No. | Sample | tration (%) | time (hr) | (° C.) | (Brix) |
| 1 | Enteromorpha | 50 | 2 | 60 | 15 |
| | prolifera | | | | |
| 2 | Sargassum | 50 | 2 | 60 | 15 |
| | horneri | | | | |
| 3 | Enteromorpha | 50 | 2 | 60 | 15 |
| | prolifera + | | | | |
| | Sargassum | | | | |
| | horneri | | | | |

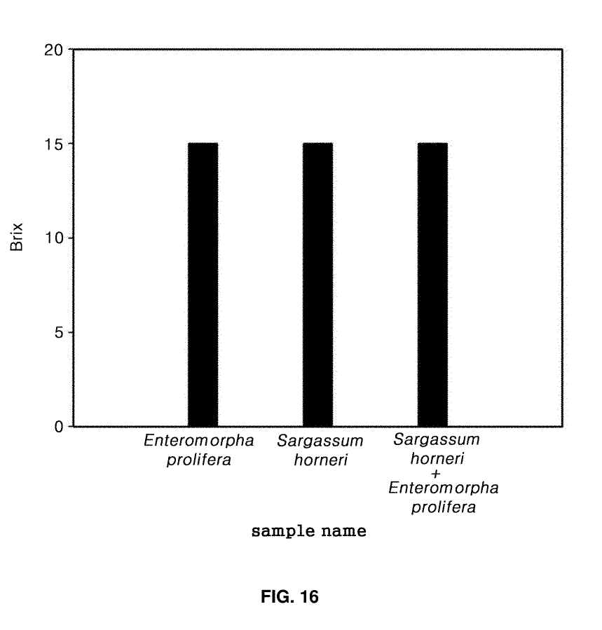

FIG. 16 is a graph showing the dependency on the extraction temperature according to a preferred embodiment of the present invention, and shows the results of Table 7. Based on the results of the extraction process of Enteromorpha prolifera and Sargassum horneri mixed at a ratio of 0.5 to 1.2:0.5 to 1.2 (preferably 1:1) with ethanol having a concentration of 40 to 60% for 1 to 3 hr at 50 to 70° C. in accordance with the optimal conditions measured upon lab testing, all of Enteromorpha prolifera, Sargassum horneri and the mixed sample of Enteromorpha prolifera and Sargassum horneri exhibited the same sugar content of 15 Brix.

Since Enteromorpha prolifera and Sargassum horneri are almost limitlessly available from nature, they may be effectively extracted in large amounts by establishing the conditions of ethanol extraction in which the yield thereof is somewhat low but antioxidant activity is high.

<Evaluation of Cytotoxicity>

Based on the results of the aforementioned <Evaluation of antioxidant activity> and <Establishment of ethanol extraction conditions>, the complex extract obtained through ethanol extraction of the mixed sample of Enteromorpha prolifera and Sargassum horneri was measured for cytotoxicity.

Animal Cells

In order to evaluate the cytotoxicity on animal cells by the complex extract (Enteromorpha prolifera+Sargassum horneri) under the above established conditions, an MTT assay was performed.

The MTT (3-(4,5-dimethylthylthiazol-2-yl)-2,5-diphenyl-tetrazolium bromide) assay is responsible for assessing the growth of cells, and is a test method that utilizes the capability of mitochondria to reduce a water-soluble yellow tetrazolium salt MTT into a water-insoluble formazan crystal having a purple color in viable cells.

As for B16F10 cells incubated in culture media containing the complex extracts at different concentrations for 24 hr, an MTT reagent (Sigma, USA) was dissolved in phosphate-buffered saline (PBS), filtered and added at a final concentration of 0.5 mg/ml to each well, followed by incubation at 37° C. for 4 hr and then dissolution in DMSO, after which the absorbance thereof was measured at 570 nm.

TABLE 8

| | | | | | Standard |
|---|---|---|---|---|---|
| mg/ml | 1 | 2 | 3 | Mean | Deviation |
| 0 | 100.0 | 100.0 | 100.0 | 100.0 | 0.0 |
| 0.625 | 92.7 | 100.3 | 99.3 | 97.4 | 2.4 |
| 1.25 | 93.8 | 100.1 | 100.0 | 98.0 | 2.1 |
| 2.5 | 95.2 | 103.6 | 102.4 | 100.4 | 2.6 |
| 5 | 90.5 | 87.5 | 89.3 | 89.1 | 0.9 |

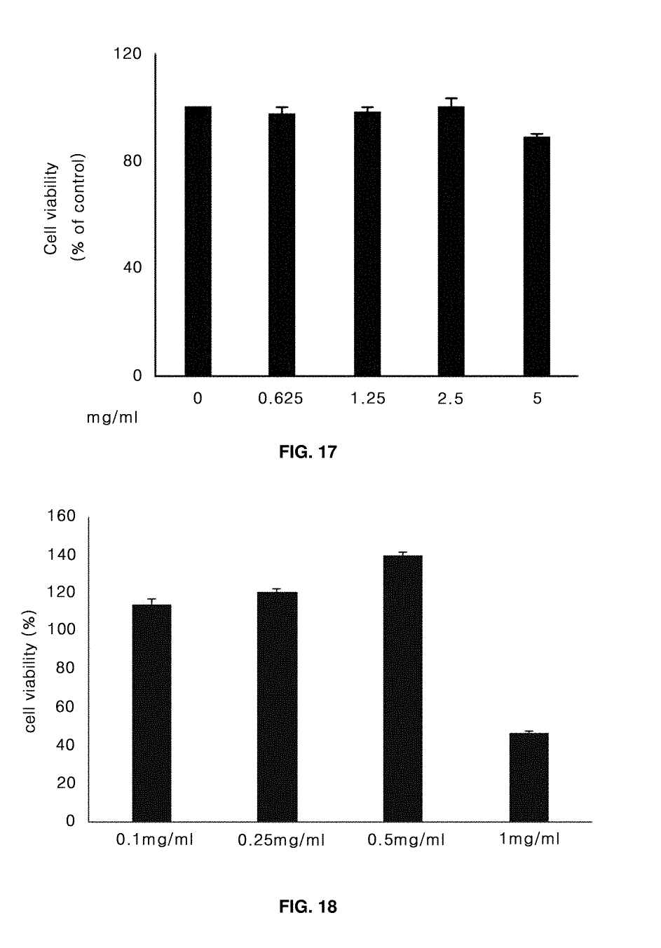

FIG. 17 is a graph showing the cytotoxicity on animal cells according to a preferred embodiment of the present invention. That is, the results of Table 8 are graphed in FIG. 17. Here, the cytotoxicity on animal cells was represented through cell viability depending on the concentration.

Human-Derived Cells

In order to evaluate the cytotoxicity on human-derived cells by the complex extract (Enteromorpha prolifera+Sargassum horneri), an MTT assay was performed. The MTT (3-(4,5-dimethylthylthiazol-2-yl)-2,5-diphenyl-tetrazolium bromide) assay is responsible for assessing the growth of cells, and is a test method that uses the capability of mitochondria to reduce a water-soluble yellow tetrazolium salt MTT into a water-insoluble formazan crystal having a purple color in viable cells.

As for CCD986-sk cells incubated in culture media containing the complex extracts at different concentrations for 48 hr, an MTT reagent (Sigma, USA) was dissolved in PBS, filtered and added at a final concentration of 0.5 mg/ml to each well, followed by incubation at 37° C. for 4 hr and then dissolution in DMSO, after which the absorbance thereof was measured at 570 nm.

TABLE 9

| | | | | | Standard |
|---|---|---|---|---|---|
| mg/ml | 1 | 2 | 3 | Mean | Deviation |
| 0.125 | 116.70 | 111.29 | 113.99 | 113.99 | 2.70 |
| 0.25 | 122.47 | 118.44 | 120.45 | 120.45 | 2.02 |
| 0.5 | 141.47 | 137.65 | 139.56 | 139.56 | 1.91 |
| 1 | 45.78 | 47.58 | 46.68 | 46.68 | 0.90 |

FIG. 18 is a graph showing the cytotoxicity on human-derived cells according to a preferred embodiment of the present invention. That is, the results of Table 9 are graphed in FIG. 18. Here, the cytotoxicity on human-derived cells was represented through cell viability depending on the concentration. For reference, 100 or more designates cell proliferation and 100 or less designates cell death, on the basis of 100.

Based on the results of the cytotoxicity test, the complex extract obtained through ethanol extraction of the mixed sample of Enteromorpha prolifera and Sargassum horneri was found not to exhibit toxicity for cell viability.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

Thus, the embodiments of the present invention do not limit the spirit of the invention but are provided to explain it, and the scope of the present invention is not limited by such embodiments.

Furthermore, it is to be understood that the scope of protection of the invention is set forth by the following claims, and all the technical ideas within the range equivalent thereto are incorporated into the scope of the invention.

What is claimed is:

1. A cosmetic composition, containing, as an active ingredient, a complex extract of Sargassum horneri and Enteromorpha prolifera.

2. The cosmetic composition of claim 1, wherein the complex extract has a total polyphenol content of 180 to 345 mg/g.

3. The cosmetic composition of claim 1, wherein the complex extract is obtained by mixing Sargassum horneri with Enteromorpha prolifera and then performing ethanol extraction.

4. The cosmetic composition of claim 3, wherein the complex extract is obtained through extraction with ethanol having a concentration of 40 to 60% at 50 to 70° C. for 1 to 3 hr.

5. The cosmetic composition of claim 3, wherein the complex extract comprises Sargassum horneri and Enteromorpha prolifera, which are mixed at a weight ratio of 0.5 to 1.2:0.5 to 1.2.
